(19) United States
(12) Plant Patent Application Publication
(10) Pub. No.: US 2014/0096294 P1
(43) Pub. Date: Apr. 3, 2014

(54) Black berry plant named: 'Willamette Thornless Marion'

(50) ;

(71) Applicant: Leonard Joseph Heidt, Mt. Angel, OR (US)

(72) Inventor: Leonard Joseph Heidt, Mt. Angel, OR (US)

(21) Appl. No.: 13/573,608

(22) Filed: Sep. 28, 2012

(51) Int. Cl. A01H 5/00

(52) U.S. Cl. PLT/203

(57) ABSTRACT

The present invention relates to a new and distinct cultivar of blackberry plant named “Willamette Thornless Marion’. The new cultivar originated as a mutation of the ‘Marion’. It is nearly identical in all ways to the ‘Marion’ except that it does not have thorns.

### REFERENCES

None Used

There was no Federally-Sponsored Research or Development.

### LATIN NAME

Rubus L. Subgenus Rubus

### VARIETY DENOMINATION

‘Willamette Thornless Marion’

### BACKGROUND OF THE INVENTION

The new cultivar ‘Willamette Thornless Marion’ is a thornless (spineless) mutation of the cultivar ‘Marion’ (non-patented). The ‘Marion’ cultivar (marketed as Marionberry) was developed by the USDA Research Service at Oregon State University in Corvallis, Oreg. The ‘Marion’ is widely produced and accounts for more than half of the blackberry production in Oregon. This new cultivar was discovered in 2005 in a field of cultivated ‘Marions’ in Clackamas County, Oregon.

### Comparison to Similar Varieties

The ‘Marion’ cultivar is a vigorously growing trailing vine. Primocanes grow the first year usually producing several canes 15 to 20 feet long. During the second year these canes then produce fruit spurs while new primocanes grow from the root of the plant. These fruit spurs develop from where the previous year's leaves have dropped off. The fruit spurs produce many berries which ripen in mid-summer.

The fruit is a bright black color whose length is about twice as long as its width. The berry has a tart sweetish flavor. The more powerful and complex flavor of the Marionberry has led to it to become the leading blackberry produced.

The main drawback with the ‘Marion’ is that the leaf stems which remain from the first year, have many thorns. During mechanical harvest, these stems fall off and attach themselves to the fruit; they then become very difficult to remove. These stems with thorns become a liability to producers, processors, and final users.

The new cultivar ‘Willamette Thornless Marion’ originated as a mutation of the ‘Marion’. It is nearly identical in all ways except that it does not have the thorns. It has the same color and flavor as the ‘Marion’, the same growing characteristics, harvests at the same time, and processes in a similar manner. The key advantage to this new cultivar is that the problem of stems with thorns during mechanical harvest is eliminated. In addition, since the primocanes do not have thorns, the care and handling of the canes is simplified, and worker fatigue and injury is significantly reduced.

### SUMMARY OF THE INVENTION

From the original thornless mutant plant, several new plants were generated asexually by tip layering, thus giving a true genetic copy of the original. After several successive generations of propagating by tip layering the plants retained their original thornless quality. As with the ‘Marion’ cultivar, in the first year after planting the canes grew vigorously and produced no fruit and reached a length of 15 to 20 feet. During the second year these canes then produced fruit while new primocanes grew from the root of the plant.

### DESCRIPTIONS OF ILLUSTRATIONS

The accompanying photographs show typical specimens of the fruit, leaves and canes of the new cultivar as well as the typical specimens of the fruit, leaves and canes of the original ‘Marion’.

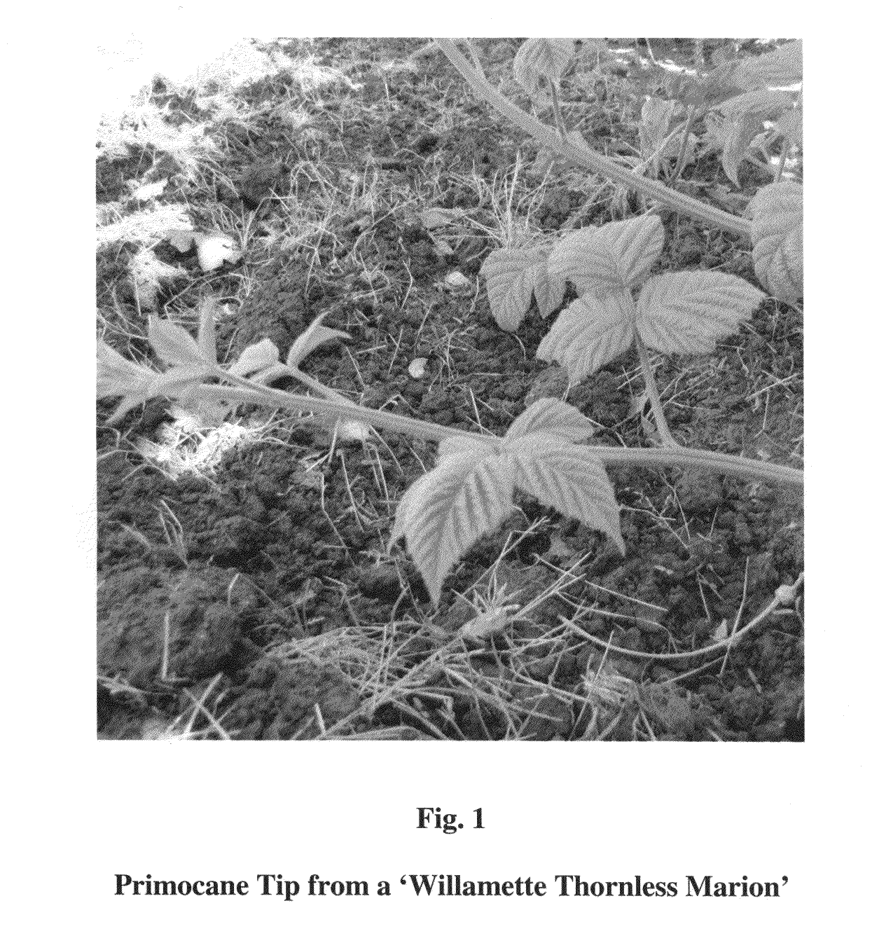

FIG. 1: A photo of the primocane tip of the new cultivar.

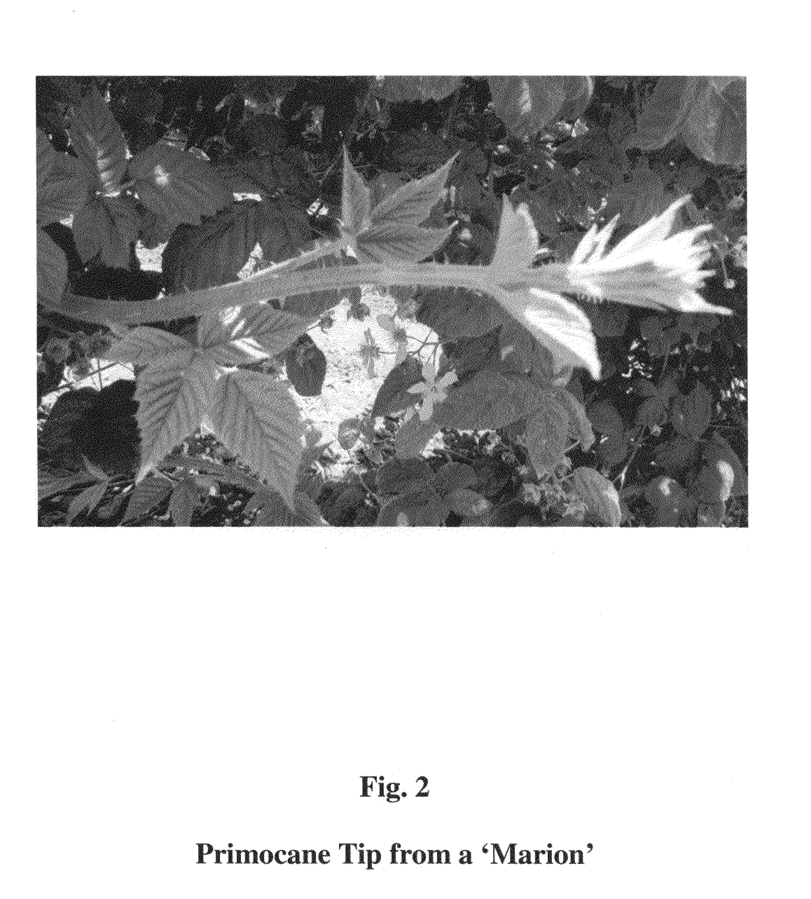

FIG. 2: A photo of the primocane tip of a ‘Marion’ blackberry.

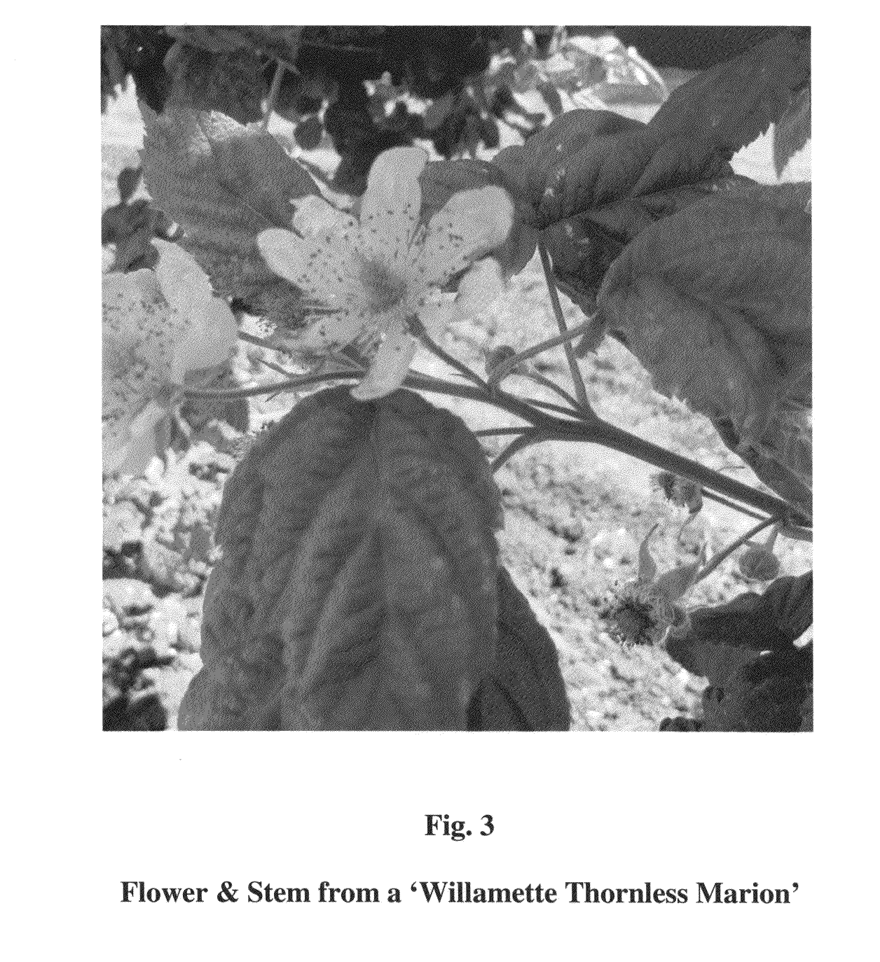

FIG. 3: A photo of the flower and stems of the new cultivar.

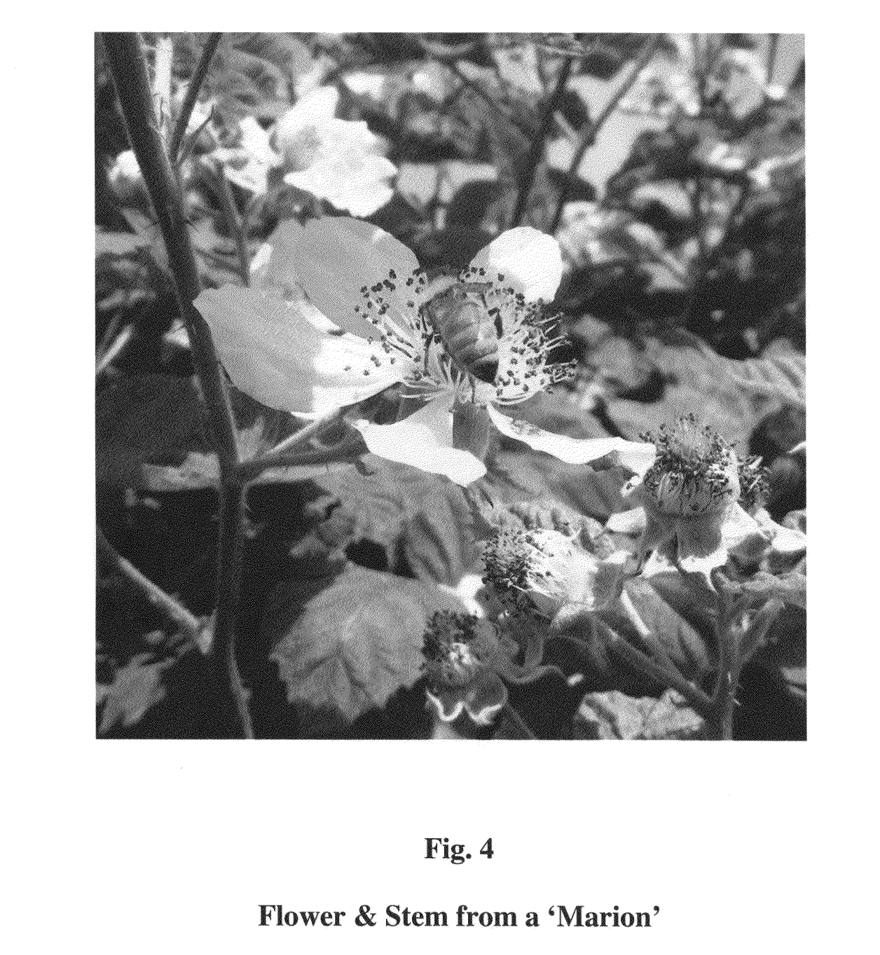

FIG. 4: A photo of the flower and stems of a ‘Marion’ blackberry.

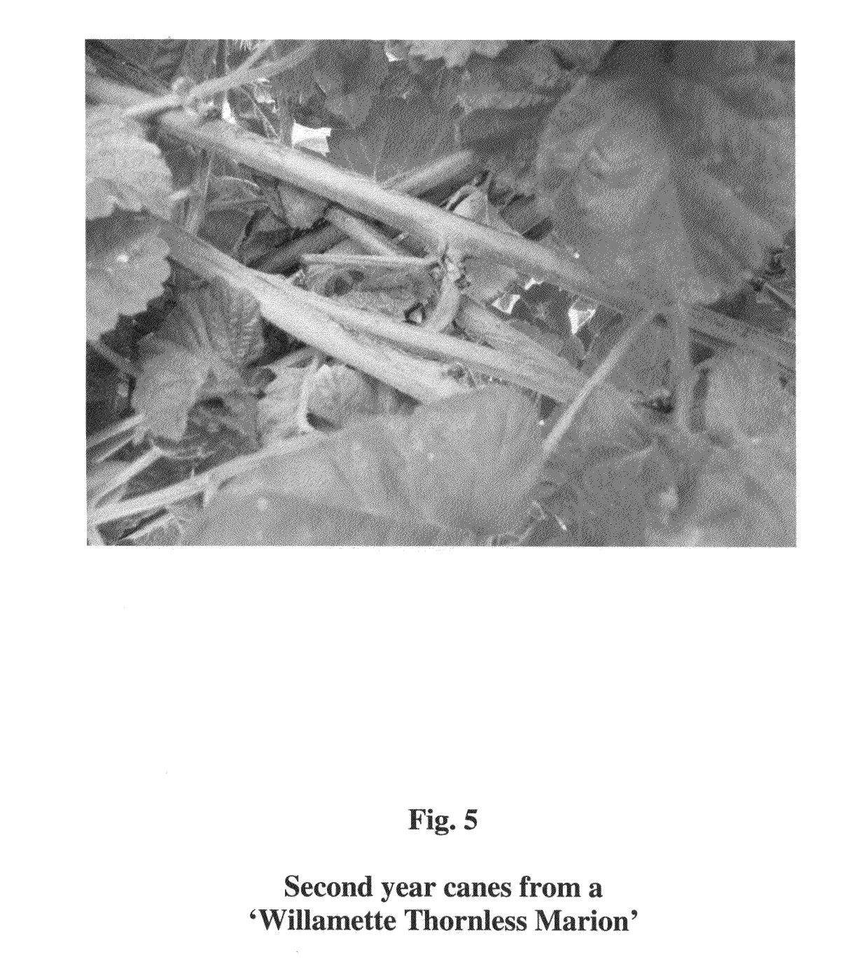

FIG. 5: A photo of the second year canes of the new cultivar.

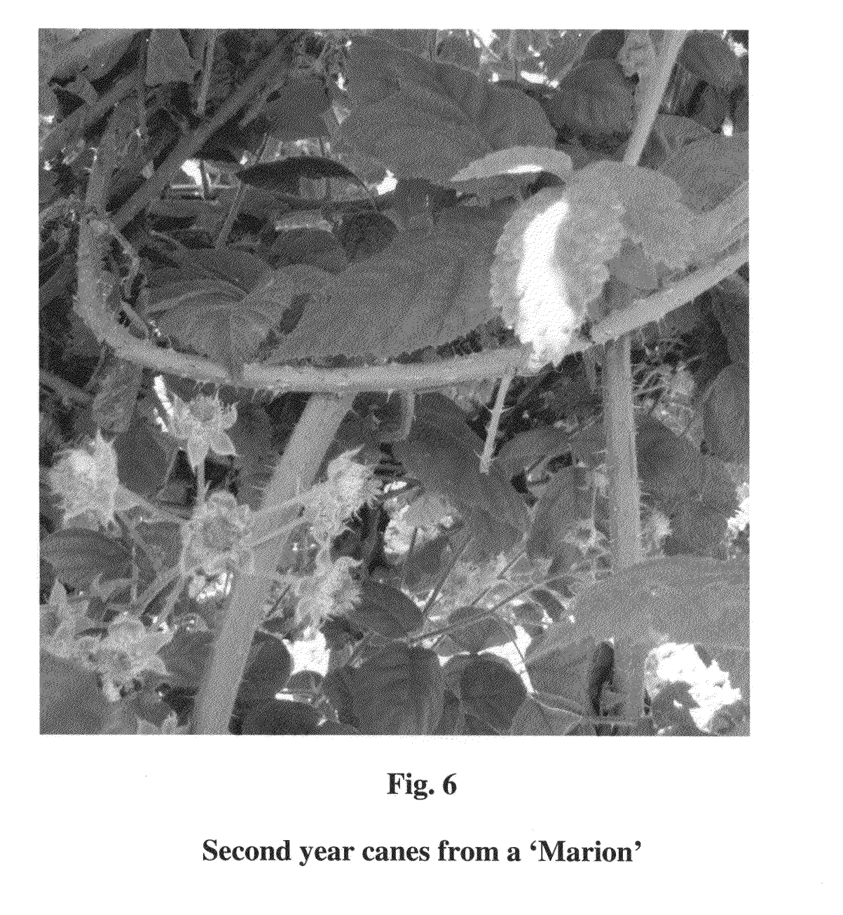

FIG. 6: A photo of the second year canes of a ‘Marion’ blackberry.

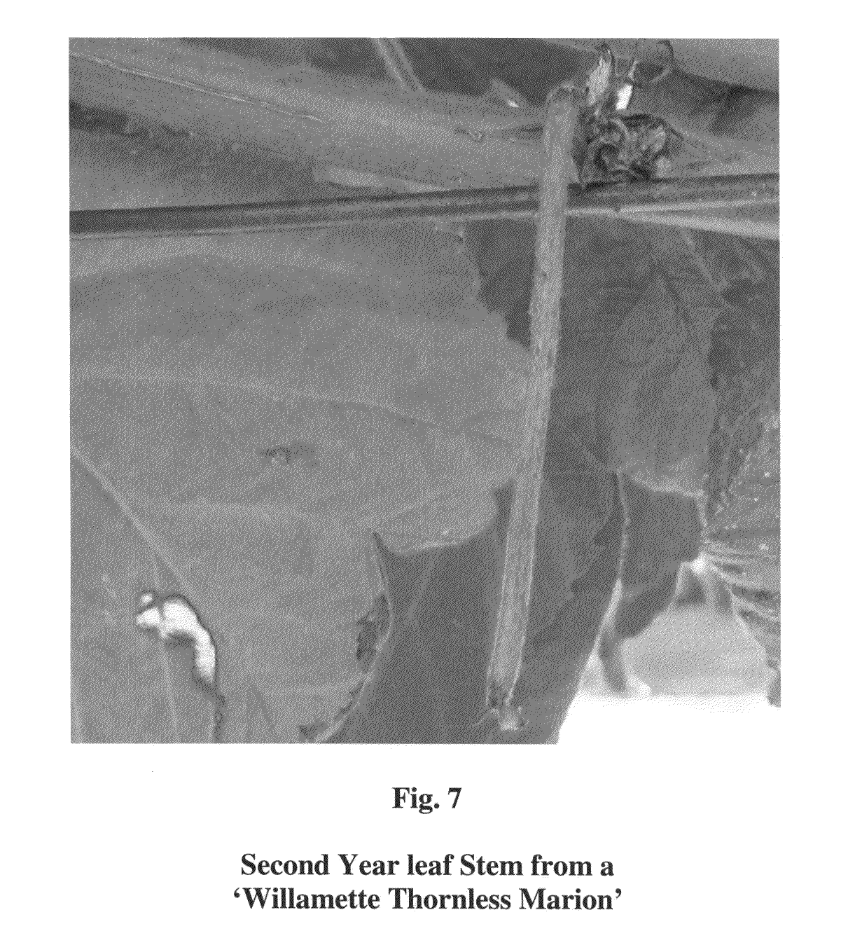

FIG. 7: A photo of a leaf stem from the new cultivar.

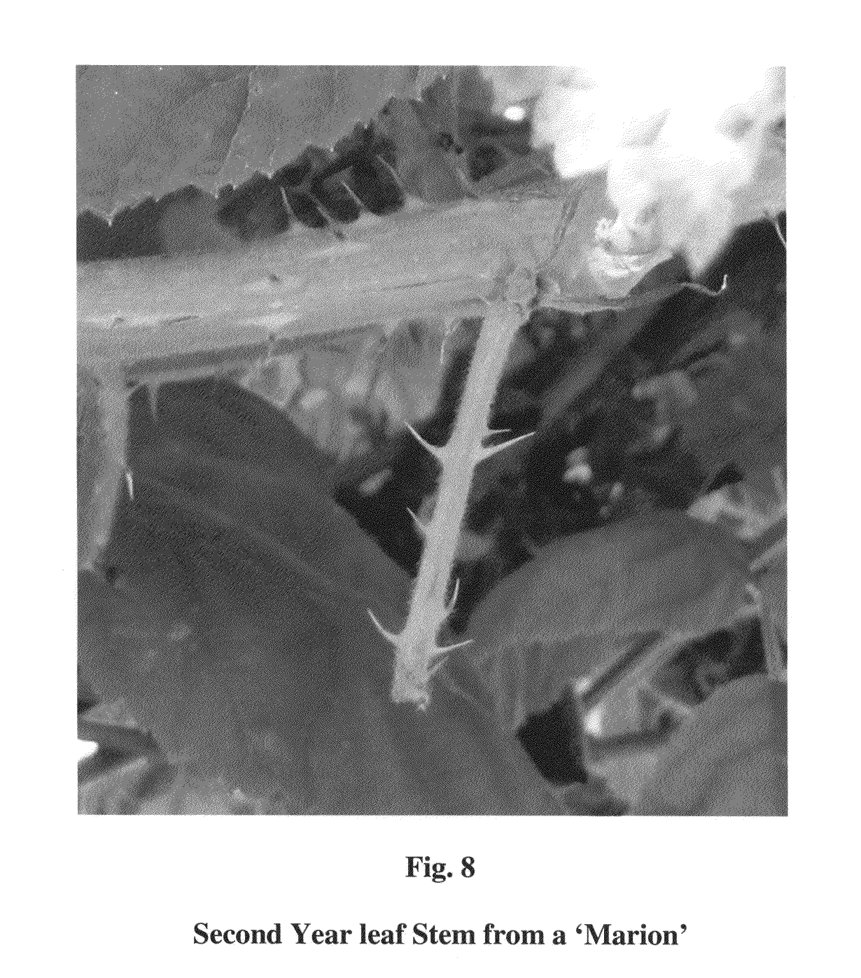

FIG. 8: A photo of a leaf stem from a ‘Marion’ blackberry.

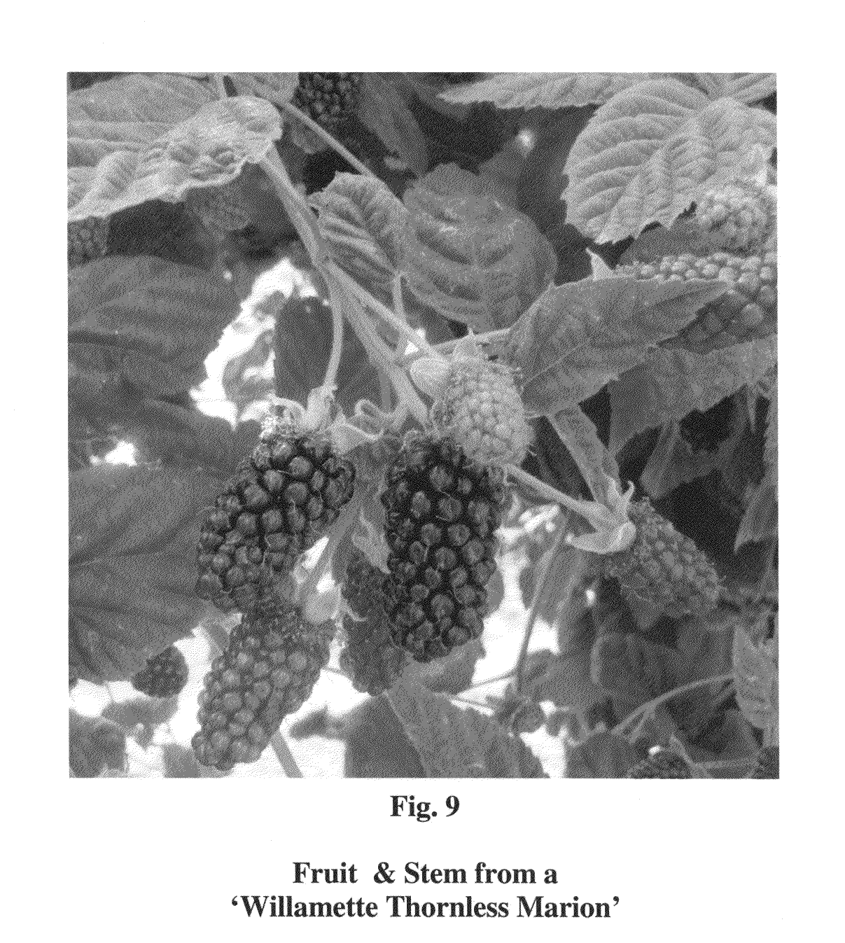

FIG. 9: A photo of the fruit and stems of the new cultivar.

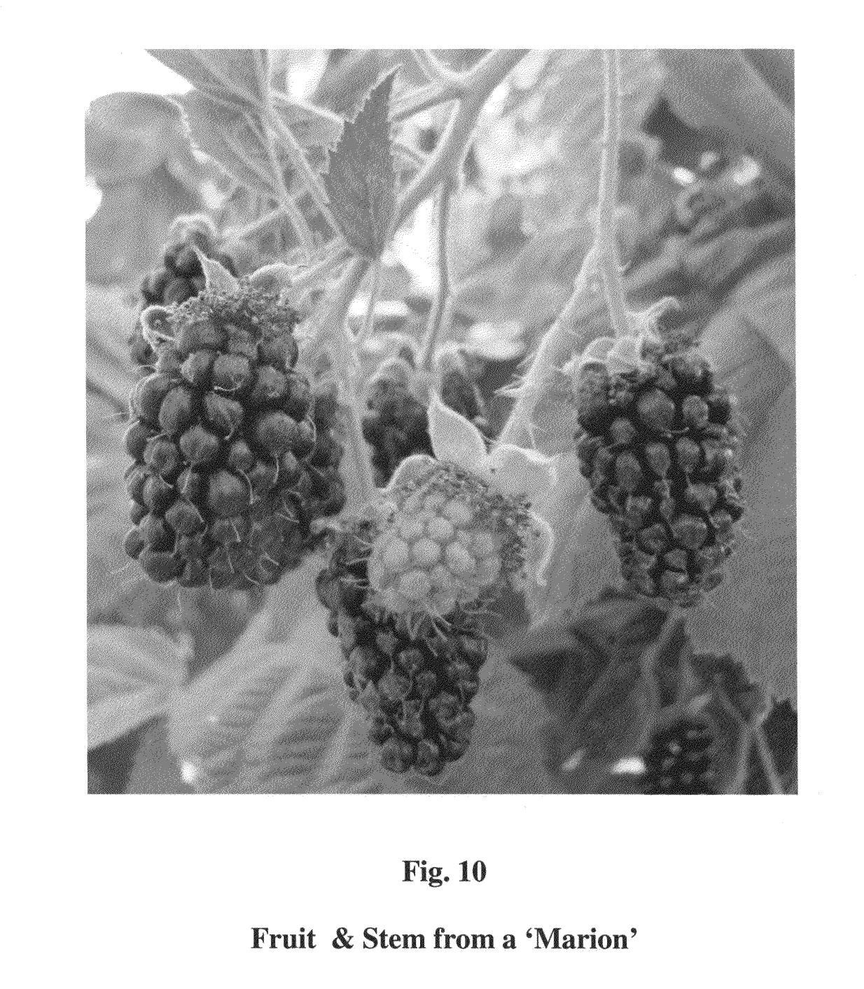

FIG. 10: A photo of the fruit and stems of a ‘Marion’ blackberry.

### BOTANICAL DESCRIPTION OF THE PLANT

The descriptions are for the plant grown in the Willamette Valley in Oregon from several generations of the same plant. The dimensions, sizes, colors and other characteristics are averages not specifics.
Plant:
   Canes.—The plant produces from five to eight vigorous primocanes from the root stock each growing season. These canes reach the length of 15 to 20 feet by the end of the growing season, usually in October. The canes begin with short and soft thorns as it comes out of the ground in early spring, then once active growth begins in warmer weather the thorns do not grow, however, occasionally soft thorns show up along the new cane. The cane diameter varies between ⅜ inch and ¾ inch. All canes are green in color.
   Foliage.—The leaves appear about every 1½ to 3 inches on the new canes and are 4 inches in length and 2 inches wide with 3 per stem.
   Flowers.—The average is between 7 and 10 per fruit spur with the average diameter of flower at 1¾ inches. The flowers are white with a pinkish hue. Bloom time is from mid-May through mid-June. Each bloom last for a period of one to two days.
   Fruit.—The fruit matures 6 to 7 weeks after the bloom is finished. The fruit is a bright black color whose length is about twice as long as its width. The fruit taste is the same as the ‘Marion’ berry which has a tart sweetish flavor. It is larger, sweeter and juicier than the most blackberries.
   Reproduction.—The plant is asexually reproduced by tip layering, that is by placing the tip of the primocane in the ground in the fall after it quits growing, then covering it with 2 inches of soil. Then in the following March it is clipped free of the primocane and then can be planted when the new shoot is 3 inches or more in length usually in May. Each primocane will produce from 10 to 20 new tips for layering each season. Plants may be reproduced by micro propagation for large numbers.

1. This is a new and distinctive cultivar of trailing blackberry originating from the ‘Marion’ berry and described herein.
